(19) United States
(12) Patent Application Publication
(10) Pub. No.: US 2007/0259431 A1
(43) Pub. Date: Nov. 8, 2007

(54) Optical Sensor and Methods of Making It

(76) Inventors: Steven C. Charlton, Oceola, IN (US); Dijia Huang, Granger, IN (US); Suny J. George, Granger, IN (US); Sung-Kwon Jung, Granger, IN (US)

Correspondence Address: NIXON PEABODY LLP, 161 N. CLARK STREET, 48TH FLOOR, CHICAGO, IL, 60601, US

(21) Appl. No.: 11/661,841

(22) PCT Filed: Sep. 19, 2005
(86) PCT No.: PCT/US05/33653

§ 371 Date: Mar. 5, 2007

Related U.S. Application Data
(60) Provisional application No. 60/611,464, filed on Sep. 20, 2004.

(51) Int. Cl. G01N 33/66; B01L 11/00; B05D 5/12

(52) U.S. Cl. 436/2; 427/58; 73/61.41

(57) ABSTRACT

A sensor for optically measuring an analyte contained in a liquid biological sample, particularly measuring the glucose content of blood in a glucose meter. The sensor in a preferred embodiment takes the form of a snow-boot. That is, it has a top portion including an air vent and an area that the user grasps to insert and remove the sensor from the slot in a glucose meter. The bottom or toe region of the sensor extends from the glucose meter and provides the entrance to a capillary channel for introducing a sample of blood into the meter, where it contacts reagents providing an optical response. The optics within the meter read the optical response of the reagents and correlates it with the glucose content of the sample.

### CROSS-REFERENCE TO RELATED APPLICATION

This application claims priority to U.S. Provisional Application No. 60/611,464, filed on Sep. 20, 2004.

### FIELD OF THE INVENTION

This invention relates generally to the field of medical devices. More particularly to devices used by a patient, rather than a medical professional.

### BACKGROUND OF THE INVENTION

The invention concerns analysis of biological samples, such as blood, urine and the like, carried out by an individual for determining the status of their body chemistry. Instruments have been developed to allow frequent testing at home or at other places without the need to submit samples to medical laboratories. For example, the invention concerns diabetic individuals who must test their blood frequently to determine the glucose content, so that their diet and medication can be adjusted. Although the invention will be described in relation to measuring glucose in blood, it has application to measuring other analytes such as cholesterol, HDL-cholesterol, triglycerides, and fructosamine.

The methods used may be generally divided into optical and electrochemical methods. Using either type of sensor requires contacting a liquid biological sample with reagents and then measuring the response, that is, an optical response such as color or fluorescence, or an electrical current produced by application of a potential to electrodes in contact with the reagents. The present invention is directed in one embodiment to optical methods in which a blood sample is brought into contact with dry reagents, producing a response that is detected by optics provided in a glucose meter. Such optical systems, which can produce more accurate results and are less expensive than electrochemical systems, are particularly attractive methods for the frequent monitoring of blood glucose content.

Each test requires a new sensor, therefore the sensor must be inexpensive and yet be able to provide accurate results. It will be appreciated that the sensors, even though they are inexpensive, must be precision devices. All of the parameters that control the amount of color generated by the analyte must be carefully controlled during manufacture. The amount of the reagents must be the same in each sensor and their response to the presence of glucose or other analyte must be uniform. It follows that sensors must be tested as manufactured and that the characteristics of each sensor be provided to the instrument with which the sensors are to be used, so that accurate results are reported to the patient.

Color changes developed by chemical reactions with the glucose in blood can be measured optically by several types of instruments, including diffuse reflectance, transmittance, absorbance, diffuse transmittance, total transmittance and the like. For example, diffuse reflectance is used in the methods described in U.S. Pat. Nos. 5,611.999 and 6,181,417. Light from light emitting diodes (LEDs) is directed onto a substrate that has been in contact with whole blood and has developed an optically measurable response. Reflected light is directed to a photo detector where the amount of light received is measured and correlated with the amount of glucose in the blood sample.

Several types of chemical reactions have been used to cause a change that is detectable by optical instruments. These include reacting glucose with glucose oxidase or glucose dehydrogenase to develop colors which indicate the quantity of glucose in the sample being tested. See for example U.S. Pat. No. 4,689,309. The present invention is not considered to be limited by the type of chemical reaction used to determine the amount of glucose in whole blood, provided only that the response to glucose in the sample is detectable by optical instruments. Nor is it limited only to glucose measurements but has application to determining the amount of other analytes in liquid biological samples.

The present inventors wanted to develop a sensor that would be inexpensive to make and use, but also would provide accurate and reliable results in the hands of non-professionals. One approach has been to provide sensors in packages that are not handled by the user, for example, the AutoDisc system from Bayer. In contrast, the present inventors wanted to avoid the complexities of packaging a group of sensors and dispensing them as required. Instead, they wanted to allow the user to grasp each sensor, insert it into an optical meter, carry out the test, and then remove and discard the used sensor. However, handling individual sensors risks contamination of the instrument and degrading the performance of the sensors. These problems have been overcome in the new sensor to be described below.

A test piece or sensor for use with a meter is described in Japanese Patent Application 1997-284880. When the sensor is inserted into the meter, the entry port for a sample and a ridged holding part extend outside the meter. A sample applied to the entry port travels inside the meter by capillary action to reach reagents that provide a response to the sample. The present invention employs some of these features, but differs in many aspects and provides advantages, as will be seen in the following summary of the invention.

### SUMMARY OF THE INVENTION

A sensor of the invention is used in optically measuring the analyte contained in a liquid biological sample, particularly the glucose content of whole blood. When inserted into a meter, one end of the sensor extends from the meter. Liquid samples are applied by the user to the accessible end of the sensor. The liquid sample travels by capillary action into the meter where it contacts reagents that provide an optical response to the analyte in the sample. That response is read by the meter and reported to the user. The reagents are contained in a thin layer deposited within the capillary channel. The sensor of the invention includes an air vent from the capillary channel located downstream of the reagents which facilitates the movement of the liquid sample into the meter by capillary action. A handling part or tab also extends outside the glucose meter, making it convenient for the user to handle the sensor, that is, inserting it into the meter and removing it after use. Calibration of the sensors is provided to the meter by a bar code on one side of the sensor or by a laser-marked conductive pad printed on the sensor, in either case, on a portion of the sensor that extends into the meter. Proper alignment of the sensor is assured by markings on the sensor and the meter and by tabs which engage recesses in the meter.

Sensors of the invention can be made by web-based processes that are capable of producing large numbers of sensors. A base stock is punched to provide traction holes and other features of the sensor. Then, the capillary channel is formed between adhesive strips applied to the base stock, the reagents are applied to the desired region of the capillary channel, a conductive pad is printed if desired, and finally a strip of clear or opaque stock is applied to complete the sensor. The characteristic properties of the sensor are then tested and appropriate calibration information is added, after which the individual sensors are cut from the base stock.

### BRIEF DESCRIPTION OF THE DRAWINGS

FIG. 1 a plan view of a sensor of the invention.

FIG. 2a-c shows alternative views of the sensor of FIG. 1 in place in a meter.

FIG. 3 is a plan view of the snow boot sensor.

FIG. 4a and b show a meter and sensor of FIG. 3.

FIG. 5a-h illustrate a process for making the snow boot sensor of the invention.

FIG. 6a-d illustrate a second process for making the snow boot sensor of the invention.

FIG. 7 illustrates two methods of reporting calibration data to the associated meter.

### DESCRIPTION OF THE PREFERRED EMBODIMENTS

Detection of Glucose by Optical Methods

The invention will be described hereinafter with relation to an important application, that is, measuring the glucose content of whole blood by optical methods. When glucose is detected by optical methods, enzymes, such as glucose oxidase and glucose dehydrogenases, are typically used. Although the methods are similar, they use different enzymes, mediators and indicators.

When glucose oxidase is used, the glucose in a sample of blood is oxidized to gluconic acid with the release of hydrogen peroxide. The hydrogen peroxide is said to oxidize an indicator in the presence of a peroxidase to produce a measurable optical response, e.g., a color that indicates the amount of glucose in the sample. Some recent patents have suggested that the glucose is converted first to the gluconic acid and then to gluconolactone, while others have suggested that the gluconolactone is formed first and then hydrolyzed to gluconic acid. Regardless of which process sequence is correct, glucose oxidase enzymes have been used widely in dry test strips for measuring the glucose content of blood.

When glucose dehydrogenase enzymes are used, a co-factor is included e.g. NAD or PQQ, an indicator and a mediator, such as a diaphorase enzyme or an analog. The co-factor is reduced in the presence of the enzyme and the glucose is oxidized to gluconic acid or the gluconolactone as described above. Thereafter, the reduced co-factor is oxidized by the diaphorase or an analog thereof. In this process, an indicator such as a tetrazolium salt is reduced to produce a colored derivative, which can be measured and correlated with the amount of glucose in the sample being tested.

In the present invention, either of these methods or other chemistries may be employed, since the invention is directed to the design of a sensor for use in making optical measurements, rather than to the chemistry employed in the sensor.

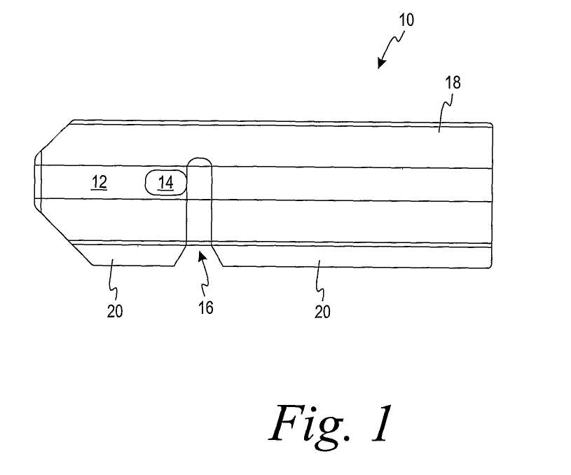

A sensor 10 of the invention is illustrated in FIG. 1. Those features include a short capillary channel 12 that conducts a blood sample to the reagents 14 contained at one end of the capillary channel and one or more vents 16 as needed to remove air displaced from the capillary channel 12 as the blood moves to the reagents 14. A handling area 18 is provided so that the user can place the sensor inside the glucose meter without coming into contact with the capillary channel or the air vent. Calibration information is provided to enable the glucose sensor to provide the necessary corrections to the glucose readings it takes. Finally, alignment features, such as tab 20, are provided to assure that the sensor is properly placed within the glucose meter. In one aspect, the invention includes methods of making the sensors, to be described below.

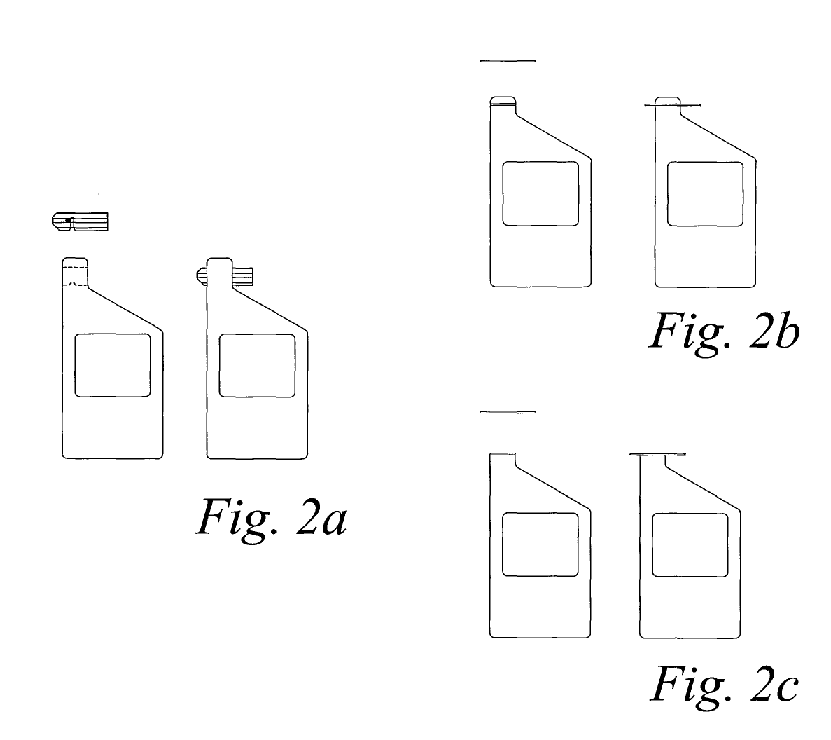

In one embodiment the glucose meter is provided with a slot for receiving the sensor. As positioned in the glucose meter, portions of the sensor will extend outside the meter, as is illustrated in FIGS. 2a-c. When it is in place, a portion of the handling area can be seen outside the meter body. The handling area makes it easier for the user to place the sensor into the meter and to retrieve it after a glucose reading has been taken. The capillary channel extends outside the meter so that the user can place a drop of blood at the end of the capillary. The blood travels by capillary action inside the meter, where it contacts the reagents, providing an optical response that is read by the optics within the meter. Air is expelled through the air vent(s) that preferably extend outside the meter. As shown in FIG. 2b, the slot is positioned so that the sensor is vertical relative to the plane of the meter. In FIG. 2a the slot is horizontal relative to the plane of the meter. Another embodiment is illustrated in FIG. 2c. No slot is provided, but instead the sensor is placed at the end of the meter, being held in place by clips, slots, and the like.

The capillary passageway is kept as short as possible in order to minimize problems with adequately filling the capillary and to minimize the amount of blood the user must supply. In one embodiment the capillary is about 0.4 inches (10.2 mm) long and has a cross-sectional area of about 0.074 mm², and thus accommodating a blood sample volume of about 0.8 μL. When positioned within the glucose meter, about one-half of the capillary will extend outside the meter. In this embodiment about 0.2 inches (5.1 mm) will protrude from the meter, which is sufficient to avoid contamination of the meter when the user deposits blood at the inlet of the capillary.

As discussed above, the reagents will be deposited at the end of the capillary so that they are in a position that is properly aligned with the optical elements of the meter. The reagents may be placed in a suitable substrate, such as polyethylene terephthalate (PET) or polycarbonate. In one embodiment, the reagents are printed onto PET. Thus, the reagents are found enclosed within the capillary passageway in the form of a thin strip, which may be about 0.0002″ (5 μm) to 0.001″ (25 μm) thick.

The sensor of the present invention differs from the design disclosed in Japanese patent application 1997-284880 in important aspects related to the design and performance of the sensor. The Japanese sensor design provides an entry port filled with an absorbent pad, while the present invention avoids the use of absorbent pads and introduces a sample directly, a method only suggested as an alternative in the Japanese design. Further, in the Japanese sensor design the liquid sample is transferred to another absorbent pad containing reagents. That pad is exposed in the sensor and is less capable of providing reliable test results than the thin layer containing reagents that in the present invention is enclosed with the capillary channel. Generally, absorbent pads have been found by the present inventors to be inferior for several reasons and consequently are not used in the present invention. Since the reagents are enclosed, contamination of the meter is avoided. Exhausting displaced air through the vent, preferably extending outside the meter, also assists in avoiding contamination of the meter, also assists in avoiding contamination of the meter.

“Snow-Boot” Glucose Sensor

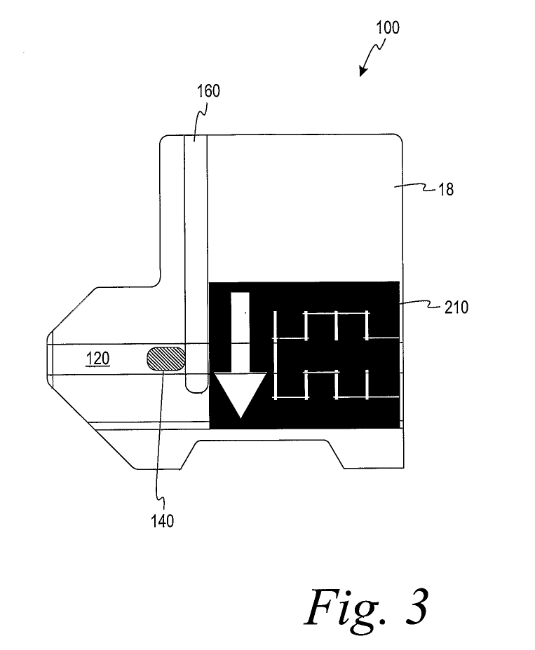

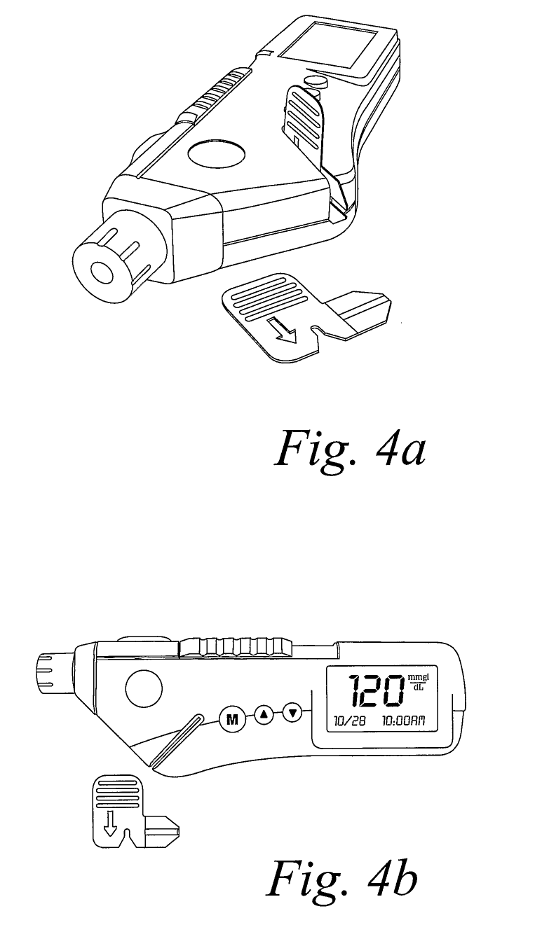

Another sensor of the invention is illustrated in FIG. 3. In the configuration shown, the sensor 100 has been called a “snow-boot” sensor because of its shape. Other embodiments may look less like a snow-boot, such as the sensor of FIG. 1, although the essential features remain. Those features include a short capillary channel 120 that conducts a blood sample to the reagents 140 contained at one end of the capillary channel 120 and one or more vents 160 as needed to remove air displaced from the capillary channel as the blood moves down the capillary channel to the reagents. The snow-boot sensor differs in that the handling area 18 is located on the side of the sensor rather than at the end aligned with the sample entry port. As with the sensor of FIG. 1, the user can place the sensor inside the glucose meter without coming into contact with the capillary channel or the air vent. Calibration information 210 is provided below the handling region to enable the glucose sensor to provide the necessary corrections to the glucose readings it takes. Finally, alignment tabs 200 are provided to assure that the sensor is properly placed within the glucose meter. In FIG. 4, the snow boot sensor is shown positioned in a glucose meter. In one aspect, the invention includes methods of making the snow-boot sensors, to be described below. These methods could be adapted to making other sensors of this type, such as the sensor shown in FIG. 1.

Making the Snow-Boot Sensor

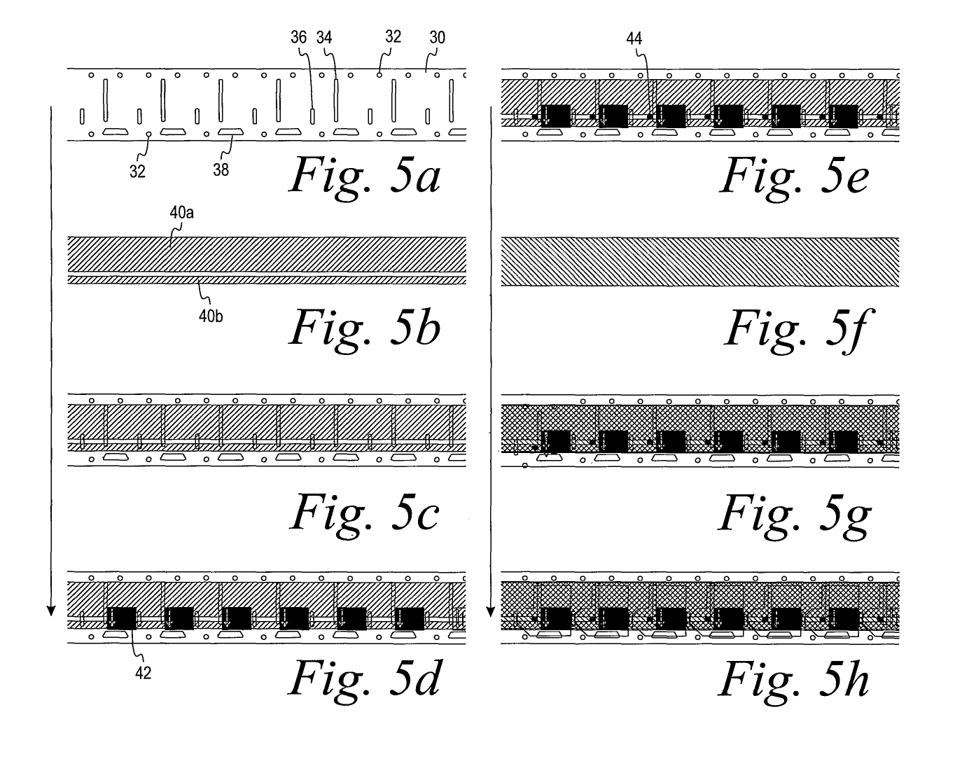

In one method of making the snow-boot sensor, shown in FIG. 5a, a base material 30 such as PET is punched to provide tractor holes 32 used to move the base stock through the various manufacturing steps and to keep the features of the sensor in registration. The longer of the vertical slots 34 will form the air vent, while the shorter vertical slot 36 will become the opening in the capillary through which a blood sample will pass. The trapezoidal hole 38 is the precursor for the tabs shown in FIG. 3, which will assure that the reagents are properly positioned when the sensor is placed in the glucose meter. The capillary channel (120 in FIG. 3) is formed by placing two ribbons 40 a and b of double-faced tape (FIG. 5b) on the base stock as shown in FIG. 5C. The space between the two ribbons defines the width of the capillary channel, which may be varied as desired. In one embodiment the two ribbons of tape are about 0.080 inches apart (2 mm). The height of the channel will be determined by the thickness of the tape, e.g. 0.004 (0.1 mm), although other thicknesses may be used. Alternatively, the capillary channel may be die-cut into the double-faced adhesive tape rather than being the space between two adhesive strips. The assembled tape is shown in FIG. 5C.

A conductive label 42 (optional) may be printed onto the spacer ribbons on the opposite side of the air vent as shown in FIG. 5d. This label will be used to provide calibration information to the glucose meter.

The reagents 44 are then deposited at the end of the channel at the entrance to the air vent (FIG. 5C). The capillary channel is completed when the exposed features of the sensor are laminated to a lid strip of PET (5f) covering the spacer ribbons, as shown in 5g. Preferably, the lid strip is treated to be hydrophilic to assure rapid filling of the sensor. At this time the sensor is completed and can be tested to determine the calibration parameters to be encoded on the label. The testing may include reaction of test sensors with blood or other appropriate calibrating solutions. The encoding could involve cutting line segments with a laser or by printing a bar-code, as will be discussed further below. After the encoding is completed, the individual sensors (seen in 5h) are cut from the web, using the traction holes for registration. Preferably, the lid strip is cut so as to extend slightly beyond the end of the capillary channel, in order to prevent the capillary channel from being closed by contact with the user's skin. This can be seen in FIG. 3.

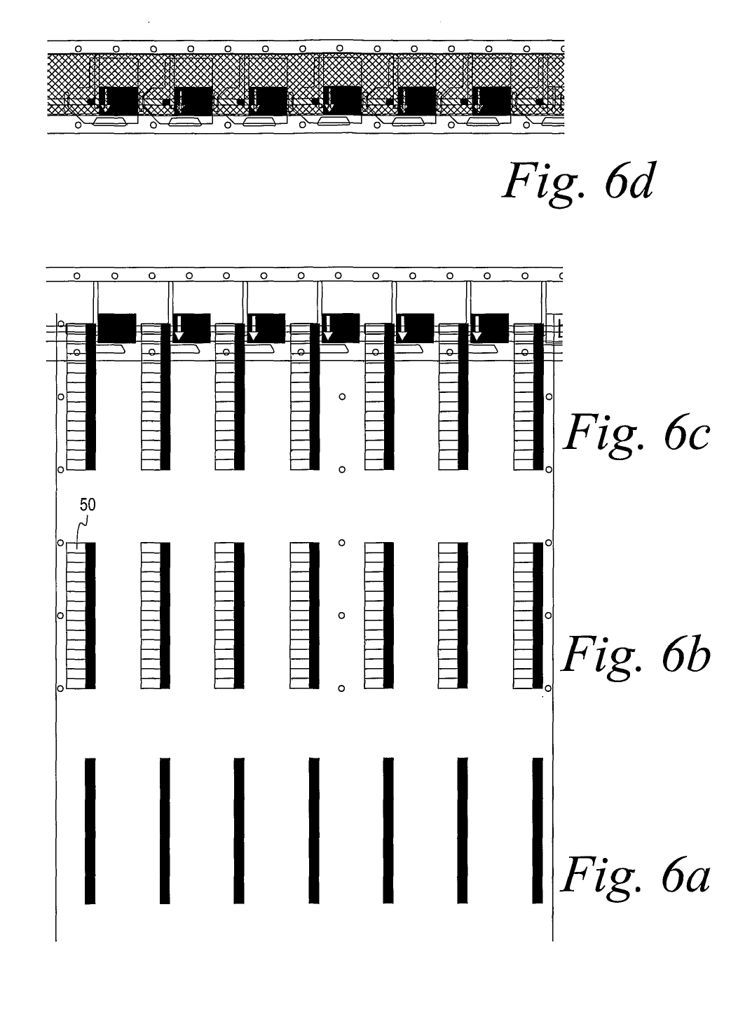

Another method of assembling the snow-boot sensor is shown in FIG. 6. In this method, the reagents are stripe coated (6a) into a substrate, which has been laminated to an easily removable temporary carrier. The reagent and its substrate are cut (6b) into segments 50 sized to be inserted into a hole punched in the base stock at the end of the capillary channel where it joins the air vent. Then, the segments containing the reagents and their substrate are brought into registration with the holes in the base stock (6c) and the temporary carrier pulled away, leaving the reagent on its substrate within the capillary channel (6d). The lid strip can then be applied, as described above.

As previously mentioned a conductive label can be used to carry calibration information to the glucose sensor. This is needed to further improve accuracy of the glucose reading, since owing to normal manufacturing tolerances in processing conditions and raw materials, each set of sensors produced is likely to provide different reading, caused by variations in the reagents. The blank label preferably is printed onto the base stock using a carbon-based conductive ink such as DuPont 7102. Alternatively, DuPont 5089, a carbon-silver conductive ink, could be used.

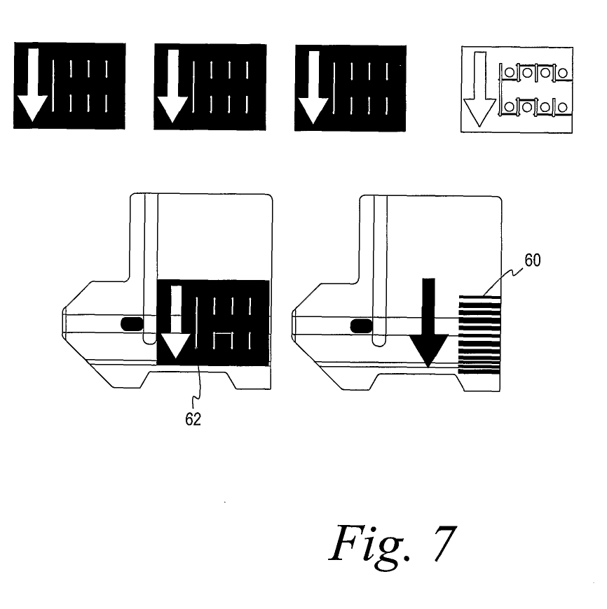

Two methods of encoding calibration information are shown in FIG. 7. In one method, a bar code 60 following U.S. Standard Bar-Code symbology or another suitable system would be printed after the sensor had been assembled and tested. In this alternative it is not necessary to apply a conductive label as discussed above. When the conductive label is used, insulating lines are laser cut vertically 62. The meter then would contain contacts to read the code on the label. This later method is described in U.S. Pat. No. 5,856,196.

Using the Sensor

Typically, a group of sensors will be supplied to the user in a container where they are protected from contamination and from ambient humidity by a desicant. When the user is ready to test their blood for its glucose content, they will extract a sensor using the handling tab and insert the sensor into a slot or other retaining means in the glucose meter, aligning the sensor appropriately. This may be done easily by matching the arrow imprinted on the sensor with an arrow on the meter. The tabs at the bottom of the sensor also assure that the reagent area is positioned properly with respect to the optics within the meter. Once the sensor is in place, the user will prick their finger to produce a drop of blood and apply it to the exposed end of the capillary channel. The blood flows into the channel by capillary action and reacts with the reagents. The colormetric response is measured by the meter and converted by a suitable algorithm into a reading of the glucose content of the blood sample, i.e. as mg/dL.

Other Applications

In addition to its use in measuring the glucose content of whole blood, the sensor of the invention may be adapted to other uses, where a liquid biological sample, including but not limited to urine, plasma, or saliva, is added to the sensor, brought inside the associated meter and contacted with reagents to provide an optical response.

Alternative Embodiment A

A sensor for use in optically measuring in a meter the analyte contained in a liquid biological sample, said sensor having portions extending beyond said meter when in use and comprising:

(a) a capillary channel extending outside said meter for accepting and transferring said liquid biological sample into said glucose meter through a capillary channel;

(b) reagents disposed within said capillary channel for reacting with said liquid biological sample and providing an optical response;

(c) at least one air vent disposed downstream of said reagents for relieving air displaced by said liquid biological sample from said capillary channel;

(d) a handling area extending outside said meter and adjacent said air vent for inserting and removing said sensor from said meter;

(e) means for providing encoded information to said meter; and

(f) means for aligning said sensor with said meter.

Alternative Embodiment B

The sensor of embodiment A wherein said sensor is substantially flat and adapted to be inserted into a slot in said meter with portions of said capillary channel, said air vent and said handling area extending outwardly from said slot.

Alternative Embodiment C

The sensor of embodiment B wherein said sensor is substantially flat and in the shape of a snow-boot adapted to be inserted into a slot in said meter with portions of said capillary channel, said air vent and said handling area extending outwardly from said slot.

Alternative Embodiment D

The sensor of embodiment A wherein said sensor is substantially flat and adapted to be positioned adjacent said meter with portions of said capillary channel, said air vent and said handling area extending outwardly from said meter.

Alternative Embodiment E

The sensor of embodiment A wherein said means for providing encoded information is a bar code.

Alternative Embodiment F

The sensor of embodiment A wherein said means for providing encoded information is a laser encoded conductive pad.

Alternative Embodiment G

The sensor of embodiment A wherein said means for aligning said sensor with said meter includes at least one tab disposed at the base of said sensor and engaging said meter.

Alternative Embodiment H

The sensor of embodiment A wherein said sensor comprises a base stock, a pair of adhesive ribbons laminated to said base stock defining said capillary channel, and a outer lid.

Alternative Embodiment I

The sensor of embodiment H wherein said outer lid is transparent.

Alternative Embodiment J

The sensor of embodiment H wherein said outer lid extends beyond the end of said capillary channel.

Alternative Embodiment K

The sensor of embodiment A wherein said analyte is glucose and said liquid biological sample is whole blood.

Alternative Embodiment L

The method of using the sensor of embodiment A comprising:

(a) grasping said sensor by its handling area and placing said sensor into a slot in said meter, said slot providing access of said reagents in said sensor to optics in said meter for reading the optical response of said reagents;

(b) placing a liquid biological sample at the entrance of said capillary channel;

(c) allowing a predetermined period of time for reaction of said sample with said reagents and producing an optical response; and

(d) reading the analyte content of said sample provided by the optics of said meter.

Alternative Embodiment M

The method of embodiment L wherein said analyte is glucose and said liquid biological sample is whole blood.

Alternative Process N

A method of making a sensor for use in optically measuring in a meter the analyte contained in a liquid biological sample, the method comprising acts of:

(a) providing a continuous strip substrate, said strip serving as a first side of said sensor;

(b) punching holes in said substrate strip, said holes including traction holes for maintaining registration of said strip, a precursor hole for tabs used for positioning said sensor in said glucose meter, and a hole defining a channel for venting air;

(c) forming a capillary channel between adhesive strips in the area of said substrate strip between said traction holes, the spacing between said adhesive strips defining the width of said capillary channel for moving a sample of blood, said capillary channel intersecting said hole for venting air;

(d) optionally, printing a conductive ink pad on said adhesive strips and drying said conductive ink;

(e) applying reagents for reacting with glucose in a blood sample at the intersection of the capillary channel and the hole for venting air;

(f) applying over said adhesive strips, said reagents, and said optional conductive pad a strip as the second side of said sensor; and

(g) cutting a completed sensor from said continuous substrate strip after step (f).

Alternative Process O

The method of process N wherein said capillary channel of (c) is formed by applying a pair of adhesive strips separated by the width of said capillary channel.

Alternative Process P

The method of process N wherein said capillary channel of (c) is formed by applying a single adhesive strip and cutting said capillary channel from said adhesive strip.

Alternative Process Q

The method of process N further comprising testing of said completed sensor and encoding calibration information derived from said testing of said sensor.

Alternative Process R

The method of process Q wherein said encoding is provided by a bar code printed on said sensor.

Alternative Process S

The method of process Q wherein said sensor includes said optional conductive pad and said encoding is provided by laser cutting of said conductive pad.

Alternative Process T

A method of making a sensor for use in measuring in a meter an analyte contained in a liquid biological sample, the method comprising acts of:

(a) providing a continuous strip substrate, said strip serving as a first side of said sensor;

(b) punching holes in said substrate strip, said holes including traction holes for maintaining registration of said strip, a precursor hole for tabs used in positioning said sensor in said meter, a hole defining a channel for venting air, and a hole for receiving reagents on a carrier;

(c) forming a capillary channel between adhesive strips in the area of said substrate strip between said traction holes, the spacing between said adhesive strips defining the width of said capillary channel for moving a sample of blood, said capillary channel intersecting said hole for venting air;

(d) optionally, printing a conductive ink pad on said adhesive strips and drying said conductive ink;

(e) applying reagents to a carrier strip; said carrier strip having a releasable backing strip;

(f) cutting segments of said carrier strip containing said reagents without cutting the releasable backing strips;

(g) placing a segment of said carrier strip containing reagents in said hole for receiving reagents on a carrier and removing said releasable backing strips;

(h) applying over said adhesive strips, said reagents, and said optional conductive pad a strip as the second side of said sensor; and

(i) cutting a completed sensor from said continuous substrate strip after step (h).

Alternative Process U

The method of process T wherein said capillary channel of (c) is formed by applying a pair of adhesive strips separated by the width of said capillary channel.

Alternative Process V

The method of process T wherein said capillary channel of (c) is formed by applying a single adhesive strip and cutting said capillary channel from said adhesive strip.

Alternative Process W

The method of process T further comprising testing said completed sensor and encoding calibration information derived from said testing on said sensor.

Alternative Process X

The method of process W wherein said encoding is provided by a bar code printed on said sensor.

Alternative Process Y

The method of process T wherein said sensor includes said optional conductive pad and said encoding is provided by laser cutting of said conductive pad.

While the present invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined in the appended claims.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1. A sensor for use in optically measuring in a meter the analyte contained in a liquid biological sample, said sensor having portions extending beyond said meter when in use and comprising:
(a) a capillary channel extending outside said meter for accepting and transferring said liquid biological sample into said glucose meter through a capillary channel;
(b) reagents disposed within said capillary channel for reacting with said liquid biological sample and providing an optical response;
(c) at least one air vent disposed downstream of said reagents for relieving air displaced by said liquid biological sample from said capillary channel;
(d) a handling area extending outside said meter and adjacent said air vent for inserting and removing said sensor from said meter;
(e) means for providing encoded information to said meter; and
(f) means for aligning said sensor with said meter.

2. The sensor of claim 1, wherein said sensor is substantially flat and adapted to be inserted into a slot in said meter with portions of said capillary channel, said air vent and said handling area extending outwardly from said slot.

3. The sensor of claim 2, wherein said sensor is substantially flat and in the shape of a snow-boot adapted to be inserted into a slot in said meter with portions of said capillary channel, said air vent and said handling area extending outwardly from said slot.

4. The sensor of claim 1, wherein said sensor is substantially flat and adapted to be positioned adjacent said meter with portions of said capillary channel, said air vent and said handling area extending outwardly from said meter.

5. The sensor of claim 1, wherein said means for providing encoded information is a bar code.

6. The sensor of claim 1, wherein said means for providing encoded information is a laser encoded conductive pad.

7. The sensor of claim 1, wherein said means for aligning said sensor with said meter includes at least one tab disposed at the base of said sensor and engaging said meter.

8. The sensor of claim 1, wherein said sensor comprises a base stock, a pair of adhesive ribbons laminated to said base stock defining said capillary channel, and a outer lid.

9. The sensor of claim 8, wherein said outer lid is transparent.

10. The sensor of claim 8, wherein said outer lid extends beyond the end of said capillary channel.

11. The sensor of claim 1, wherein said analyte is glucose and said liquid biological sample is whole blood.

12. The method of using the sensor of claim 1, comprising:
(a) grasping said sensor by its handling area and placing said sensor into a slot in said meter, said slot providing access of said reagents in said sensor to optics in said meter for reading the optical response of said reagents;
(b) placing a liquid biological sample at the entrance of said capillary channel;
(c) allowing a predetermined period of time for reaction of said sample with said reagents and producing an optical response; and
(d) reading the analyte content of said sample provided by the optics of said meter.

13. The method of claim 12, wherein said analyte is glucose and said liquid biological sample is whole blood.

14. A method of making a sensor for use in optically measuring in a meter the analyte contained in a liquid biological sample, the method comprising the acts of:
(a) providing a continuous strip substrate, said strip serving as a first side of said sensor;
(b) punching holes in said substrate strip, said holes including traction holes for maintaining registration of said strip, a precursor hole for tabs used for positioning said sensor in said glucose meter, and a hole defining a channel for venting air;
(c) forming a capillary channel between adhesive strips in the area of said substrate strip between said traction holes, the spacing between said adhesive strips defining the width of said capillary channel for moving a sample of blood, said capillary channel intersecting said hole for venting air;
(d) optionally, printing a conductive ink pad on said adhesive strips and drying said conductive ink;
(e) applying reagents for reacting with glucose in a blood sample at the intersection of the capillary channel and the hole for venting air;
(f) applying over said adhesive strips, said reagents, and said optional conductive pad a strip as the second side of said sensor; and
(g) cutting a completed sensor from said continuous substrate strip after step (f).

15. The method of claim 14, wherein said capillary channel of (c) is formed by applying a pair of adhesive strips separated by the width of said capillary channel.

16. The method of claim 14, wherein said capillary channel of (c) is formed by applying a single adhesive strip and cutting said capillary channel from said adhesive strip.

17. The method of claim 14, further comprising testing of said completed sensor and encoding calibration information derived from said testing of said sensor.

18. The method of claim 17, wherein said encoding is provided by a bar code printed on said sensor.

19. The method of claim 17, wherein said sensor includes said optional conductive pad and said encoding is provided by laser cutting of said conductive pad.

20. A method of making a sensor for use in measuring in a meter an analyte contained in a liquid biological sample, the method comprising the acts of:
(a) providing a continuous strip substrate, said strip serving as a first side of said sensor;
(b) punching holes in said substrate strip, said holes including traction holes for maintaining registration of said strip, a precursor hole for tabs used in positioning said sensor in said meter, a hole defining a channel for venting air, and a hole for receiving reagents on a carrier;
(c) forming a capillary channel between adhesive strips in the area of said substrate strip between said traction holes, the spacing between said adhesive strips defining the width of said capillary channel for moving a sample of blood, said capillary channel intersecting said hole for venting air;
(d) optionally, printing a conductive ink pad on said adhesive strips and drying said conductive ink;
(e) applying reagents to a carrier strip; said carrier strip having a releasable backing strip;
(f) cutting segments of said carrier strip containing said reagents without cutting the releasable backing strips;
(g) placing a segment of said carrier strip containing reagents in said hole for receiving reagents on a carrier and removing said releasable backing strips;
(h) applying over said adhesive strips, said reagents, and said optional conductive pad a strip as the second side of said sensor; and
(i) cutting a completed sensor from said continuous substrate strip after step (h).

21. The method of claim 20, wherein said capillary channel of (c) is formed by applying a pair of adhesive strips separated by the width of said capillary channel.

22. The method of claim 20, wherein said capillary channel of (c) is formed by applying a single adhesive strip and cutting said capillary channel from said adhesive strip.

23. The method of claim 20, further comprising testing said completed sensor and encoding calibration information derived from said testing on said sensor.

24. The method of claim 23, wherein said encoding is provided by a bar code printed on said sensor.

25. The method of claim 20, wherein said sensor includes said optional conductive pad and said encoding is provided by laser cutting of said conductive pad.
